(19) United States
(12) Patent Application Publication
(10) Pub. No.: US 2025/0281095 A1
(43) Pub. Date: Sep. 11, 2025

(54) METHOD AND APPARATUS FOR DETERMINING ABNORMAL CARDIAC CONDITIONS NON-INVASIVELY

(71) Applicant: MASSACHUSETTS INSTITUTE OF TECHNOLOGY, Cambridge, MA (US)

(72) Inventors: Collin M. Stultz, Newton, MA (US); Daphne E. Schlesinger, Cambridge, MA (US); Ridwan Alam, Cambridge, MA (US)

(21) Appl. No.: 18/863,758

(22) PCT Filed: May 11, 2023
(86) PCT No.: PCT/US2023/021844

§ 371 Date: Nov. 7, 2024

Related U.S. Application Data
(60) Provisional application No. 63/340,761, filed on May 11, 2022.

(51) Int. Cl. A61B 5/346; A61B 5/00; A61B 5/021; G16H 50/50

(52) U.S. Cl. CPC A61B 5/346; A61B 5/021; A61B 5/7221; A61B 5/7267; A61B 5/746; G16H 50/50

(57) ABSTRACT

A system and method are provided for monitoring patients with heart failure. One method may include receiving physiological data associated with a patient. The method may also include determining a data portion satisfying a signal-quality-index condition. The method may also include determining a probability value for the data portion with a model developed, for example, via machine learning using training data. The method may also include determining whether an exacerbation condition is satisfied based on the probability value, and, in response to determining that the exacerbation condition is satisfied, generating and transmitting an exacerbation alert associated with the patient.

### CROSS-REFERENCE TO RELATED APPLICATIONS

This application claims priority to U.S. Provisional Application No. 63/340,761, filed on May 11, 2022, the entire contents of which is incorporated herein by reference.

### STATEMENT REGARDING FEDERALLY SPONSORED RESEARCH

N/A.

### BACKGROUND

Heart failure (“HF”) is a severe illness wherein the heart cannot supply sufficient oxygen and nutrients to the body at normal filling pressures. HF is a global pandemic, affecting approximately 26 million people worldwide, and has a 5-year mortality rate greater than 50% in some cohorts. Improved methods for the management of patients with HF represents an important unmet need. HF remains one of the most challenging disorders to manage both in the inpatient and outpatient setting. Central to the care of patients with HF is an assessment of their cardiac pressures, which, traditionally, is something that can be reliably obtained via invasive approaches.

A parameter in the diagnosis and longitudinal care of patients with HF patient is the left ventricular filling pressure (e.g., increased left ventricular filling pressures portends poor outcomes in patients with HF). Interventions that reduce these pressures can alleviate the signs and symptoms associated with a HF exacerbation. Traditionally, left ventricular filling pressure, however, can be reliably measured via the insertion of a pulmonary artery catheter (“PAC”). In this procedure, a catheter with a pressure transducer on its end is inserted into a major vein, then navigated into the right heart through the vena cava. The catheter is then guided into the right ventricle, then into the pulmonary artery.

Unfortunately, PAC insertion can be associated with complications, ranging from benign self-limited arrhythmias to rare, but often fatal, pulmonary artery perforations. The PAC insertion procedure also cannot generally be scheduled expeditiously, particularly in the setting of a global pandemic of transmissible disease. Additionally, such measurements cannot be performed in the outpatient setting.

### SUMMARY

The present disclosure provides systems and methods for improving the management of patients with HF. For example, the disclosure relates to non-invasive methods that infer when important hemodynamic parameters are abnormal, which could guide clinical decisions when an invasive procedure cannot be performed (e.g., in the context of telemedicine). As yet another non-limiting example, the systems and methods described herein implement a method to estimate when the mean pulmonary capillary wedge pressure (“mPCWP”) is elevated above, e.g., 18 mmHg, using a readily available signal, such as, e.g., a single-lead electrocardiogram arising from a patch monitor. Thus, in one non-limiting example, systems and methods are provided for inferring elevated pulmonary capillary wedge pressures from single-lead electrocardiogram telemetry data.

Accordingly, the disclosure relates to systems and methods of detecting abnormal cardiac pressures in patients with HF. As one non-limiting example, the systems and methods can continuously or intermittently sense an electrocardiogram with a mobile device (e.g., a patch monitor, a smartwatch, etc.). The systems and methods can determine area(s) of the sensed ECG signal that have high signal-to-noise index (“SQI”) (e.g., as a measure of how noisy the signal is). The disclosure also relates to the development of a machine learning model or algorithm for real time (or near real-time) continuous prediction of cardiac pressures using the regions of the ECG signal with a high SQI. The systems and methods can further generate and transmit one or more alerts when cardiac pressures are elevated.

The systems and methods described herein provide for monitoring and detecting abnormal cardiac pressure for patients with HF. The systems and methods may be implemented outside of a healthcare setting (e.g., at-home) using existing technologies where the patient can be awake and ambulatory. Additionally, the systems and methods can provide for a passive monitoring and detection method (e.g., no requirement for the patient to actively perform an action, such as a series of breathing maneuvers). Furthermore, the systems and methods can be implemented using actual physiological data (e.g., pressure measurements) for a specific patient (as opposed to using historical clinical or health data unrelated to the specific patient).

In accordance with one aspect of the disclosure, a system is provided for detecting abnormal cardiac pressures in patients with heart failure. The system may include one or more electronic processors. The one or more electronic processors may be configured to receive physiological data associated with a patient. The one or more electronic processors may be configured to determine, from a first data segment included in the physiological data, a first data portion satisfying a signal-quality-index (“SQI”) condition. The one or more electronic processors may be configured to determine a first probability value for the first data portion with a model developed via machine learning using training data. The first probability value may indicate a probability that the first data portion is associated with an exacerbation event. The one or more electronic processors may be configured to determine whether an exacerbation condition is satisfied based on the first probability value. The one or more electronic processors may be configured to, in response to determining that the exacerbation condition is satisfied, generate and transmit an exacerbation alert associated with the patient.

In some configurations, the physiological data includes electrocardiogram (“ECG”) data collected by an ECG device associated with the patient. The ECG data may be single-lead ECG data.

In some configurations, the one or more electronic processors may be configured to receive the physiological data continuously from a remote monitoring device associated with the patient.

In some configurations, the one or more electronic processors may be configured to receive the physiological data intermittently from a remote monitoring device associated with the patient.

In some configurations, the one or more electronic processors may be configured to determine a set of data segments included in the physiological data and to determine a set of data portions from the set of data segments. Each data portion may be included in a corresponding data segment and is a representative signal of the corresponding data segment. The first data segment may be included in the set of data segments and the first data portion is included in the set of data portions.

In some configurations, the set of data segments may include a series of non-overlapping time windows.

In some configurations, the SQI condition may include a predetermined threshold of 0.5.

In some configurations, the first data segment may include a five-minute window of data from the physiological data.

In some configurations, the first data portion may include a ten-second window of data from a corresponding data segment.

In some configurations, the one or more electronic processors may be configured to determine, from a second data segment included in the physiological data, a second data portion satisfying the SQI condition and to determine a second probability value for the second data portion with the model developed via machine learning using the training data.

In some configurations, the one or more electronic processors may be configured to determine whether the exacerbation condition is satisfied based on the first probability value and the second probability value.

In some configurations, the third probability value may be a mean pulmonary capillary wedge pressure of the first probability value and the second probability value.

In some configurations, the one or more electronic processors may be configured to determine a third probability value based on the first probability value and the second probability value. The one or more electronic processors may be configured to determine whether the exacerbation condition is satisfied based on the third probability value.

In some configurations, the one or more electronic processors may be configured to determine whether the exacerbation condition is satisfied by comparing the third probability value to a pressure threshold.

In some configurations, the third probability value may satisfy the exacerbation condition when the third probability value exceeds the pressure threshold.

In some configurations, the pressure threshold may be 18 mmHg.

In some configurations, the exacerbation condition may indicate an elevated cardiac pressure that is indicative of an onset of heart failure exacerbation.

In accordance with one aspect of the disclosure, a method is provided for detecting abnormal cardiac pressures in patients with heart failure. The method may include receiving physiological data associated with a patient. The method may also include determining, with one or more electronic processors, a first data portion satisfying a signal-quality-index (“SQI”) condition. The method may also include determining, with the one or more electronic processors, a first probability value for the first data portion with a model relating the physiological data to cardiac pressure. The first probability value may indicate a probability that the first data portion is associated with an exacerbation event. The method may also include determining, with the one or more electronic processors, whether an exacerbation condition is satisfied based on the first probability value. The method may also include, in response to determining that the exacerbation condition is satisfied, generating and transmitting, with the one or more electronic processors, an exacerbation alert associated with the patient.

In some configurations, the method may include determining a set of data segments included in the physiological data and determining a set of data portions from the set of data segments. Each data portion may be included in a corresponding data segment and may be a representative signal of the corresponding data segment.

In some configurations, the method may include determining a plurality of data portions. Each data portion may satisfy the SQI condition, and the first data portion may be included in the plurality of data portions. The method may also include determining a plurality of probability values using the model relating to the physiological data to cardiac pressure. The first probability value may be included in the plurality of probability values and each probability value may indicate a probability that each data portion is associated with a corresponding exacerbation event. The method may also include determining a combined probability value based on the plurality of probability values. The method may also include determining whether the exacerbation condition is satisfied based on the combined probability value. The method may also include, in response to determining that the exacerbation condition is satisfied, generating and transmitting the exacerbation alert associated with the patient.

In some configurations, determining the combined probability value may include determining a mean pulmonary capillary wedge pressure based on the plurality of probability values.

In some configurations, determining the plurality of probability values may include determining a plurality of probability values, wherein each probability value is associated with a different data portion of the plurality of data portions.

This Summary and the Abstract are provided to introduce a selection of concepts in a simplified form that are further described below in the Detailed Description. The Summary and the Abstract are not intended to identify key features or essential features of the claimed subject matter, nor are they intended to be used as an aid in determining the scope of the claimed subject matter.

### BRIEF DESCRIPTION OF THE DRAWINGS

The following drawings are provided to help illustrate various features of examples of the disclosure and are not intended to limit the scope of the disclosure or exclude alternative implementations.

FIG. 1 schematically illustrates an example path of a catheter during a pulmonary artery catheter insertion procedure in accordance with the present disclosure.

FIG. 2 schematically illustrates a system for detecting abnormal cardiac pressures in patients with heart failure in accordance with the present disclosure.

FIG. 3 schematically illustrates a server included in the system of FIG. 2 in accordance with the present disclosure.

FIG. 4 is a flowchart of a method for detecting abnormal cardiac pressures in patients with heart failure using the system of FIG. 2 in accordance with the present disclosure.

FIG. 5 is a graph illustrating predictive value as a function of prevalence in accordance with the present disclosure.

FIG. 6A is a graph illustrating specificity trends for different sensitivity values in accordance with the present disclosure.

FIG. 6B is a graph illustrating positive predictive value trends for different sensitivity values in accordance with the present disclosure.

FIG. 6C is a graph illustrating negative predictive value trends for different sensitivity values in accordance with the present disclosure.

### DETAILED DESCRIPTION

The concepts disclosed in this discussion are described and illustrated with reference to exemplary embodiments. These concepts, however, are not limited in their application to the details of construction and the arrangement of components in the illustrative embodiments and are capable of being practiced or being carried out in various other ways. The terminology in this document is used for the purpose of description and should not be regarded as limiting. Words such as “including,” “comprising,” and “having” and variations thereof as used herein are meant to encompass the items listed thereafter, equivalents thereof, as well as additional items.

As noted above, HF is a severe illness wherein the heart cannot supply sufficient oxygen and nutrients to the body at normal filling pressures. HF remains one of the most challenging disorders to manage both in the inpatient and outpatient setting. Central to the care of patients with HF is an assessment of their cardiac pressures, which, traditionally, is something that can be reliably obtained via invasive approaches.

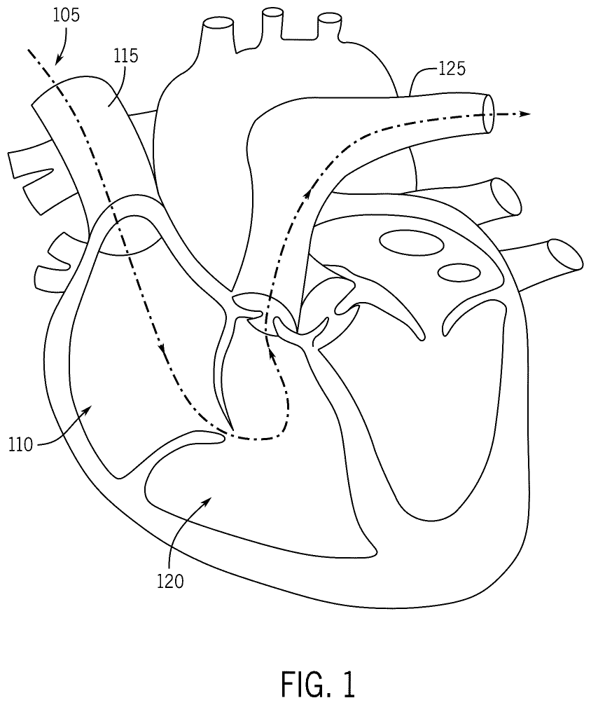

Accordingly, a parameter in the diagnosis and longitudinal care of patients with HF patient is the left ventricular filling pressure (e.g., increased left ventricular filling pressures portends poor outcomes in patients with heart failure). Interventions that reduce these pressures can alleviate the signs and symptoms associated with a HF exacerbation. Traditionally, left ventricular filling pressure can be measured via the insertion of a pulmonary artery catheter (“PAC”). FIG. 1 illustrates an example path 105 of a catheter during a PAC insertion procedure. As illustrated in FIG. 1, during a PAC insertion procedure, a catheter with a pressure transducer on its end (not illustrated) may be inserted into a major vein, and then navigated into the right heart 110 through the vena cava 115. The catheter may then be guided into the right ventricle 120, and then into the pulmonary artery 125.

As noted above, PAC insertion may be associated with complications, ranging from benign self-limited arrhythmias to rare, but often fatal, pulmonary artery perforations. The procedure also cannot generally be scheduled expeditiously, particularly in the setting of a global pandemic of transmissible disease, and these measurements cannot be performed in the outpatient setting.

Accordingly, the present disclosure provides systems and methods for improving the management of patients with HF. In one non-limiting example, systems and methods are provided for inferring elevated pulmonary capillary wedge pressures from single-lead electrocardiogram telemetry data. Accordingly, the disclosure relates to systems and methods of detecting abnormal cardiac pressures in patients with heart failure.

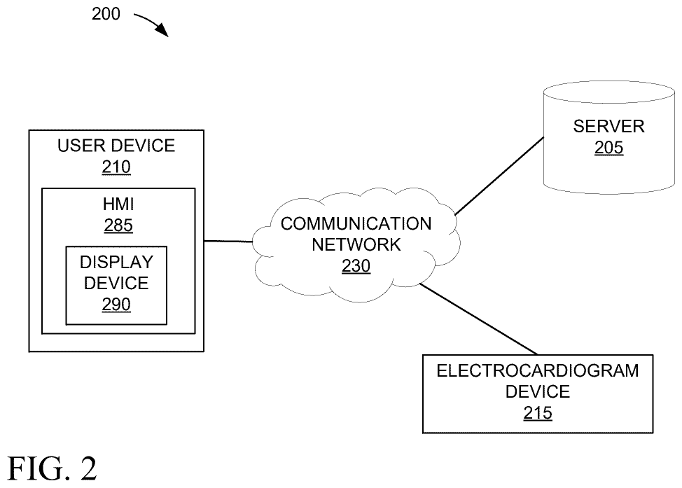

FIG. 2 schematically illustrates a system 200 for detecting abnormal cardiac pressures in patients with HF according to some embodiments. In the illustrated example, the system 200 includes a server 205, a user device 210, and an electrocardiogram (“ECG”) device 215. In some embodiments, the system 200 includes fewer, additional, or different components in different configurations than illustrated in FIG. 1. As one example, the system 200 may include multiple servers 205, user devices 210, ECG devices 215, or a combination thereof. As another example, one or more components of the system 200 may be combined into a single device, such as, e.g., the server 205 and the user device 210.

The server 205, the user device 210, and the ECG device 215 communicate over one or more wired or wireless communication networks 230. Portions of the communication networks 230 may be implemented using a wide area network, such as the Internet, a local area network, such as a Bluetooth™ network or Wi-Fi, and combinations or derivatives thereof. Alternatively, or in addition, in some embodiments, components of the system 200 communicate directly as compared to through the communication network 230. Also, in some embodiments, the components of the system 200 communicate through one or more intermediary devices not illustrated in FIG. 2.

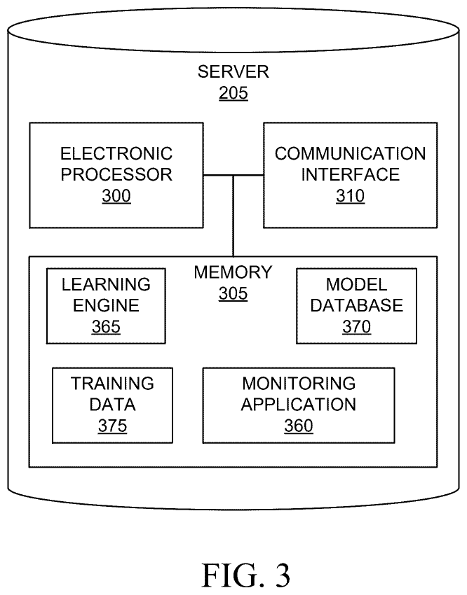

The server 205 is a computing device, such as a server, a database, or the like. As illustrated in FIG. 3, the server 205 includes an electronic processor 300, a memory 305, and a communication interface 310. The electronic processor 300, the memory 305, and the communication interface 310 communicate wirelessly, over one or more communication lines or buses, or a combination thereof. The server 205 may include additional components than those illustrated in FIG. 3 in various configurations. For example, the server 205 may also include one or more human machine interfaces, such as a keyboard, keypad, mouse, joystick, touchscreen, display device, printer, speaker, and the like, that receive input from a user, provide output to a user, or a combination thereof. The server 205 may also perform additional functionality other than the functionality described herein. Also, the functionality described herein as being performed by the server 205 may be distributed among multiple servers or devices (e.g., as part of a cloud service or cloud-computing environment), combined with another component of the system 200 (e.g., combined with the user device 210, another component(s) of the system 200, or the like), or a combination thereof.

The communication interface 310 may include a transceiver that communicates with the user device 210, the ECG device 215, or a combination thereof over the communication network 230 and, optionally, one or more other communication networks or connections. The electronic processor 300 includes a microprocessor, an application-specific integrated circuit (“ASIC”), or another suitable electronic device for processing data, and the memory 305 includes a non-transitory, computer-readable storage medium.

The electronic processor 300 can access and execute computer-readable instructions (“software”) stored in the memory 305. The software may include firmware, one or more applications, program data, filters, rules, one or more program modules, and other executable instructions. For example, the software may include instructions and associated data for performing a set of functions, including the methods described herein.

For example, as illustrated in FIG. 3, the memory 305 includes a monitoring application 360 (referred to herein as “the application 360”). The application 360 is a software application executable by the electronic processor 300 in the example illustrated and as specifically discussed herein, although a similarly purposed module can be implemented in other ways in other examples. As described in more detail herein, the electronic processor 300 executes the application 360 to perform a cardiac pressure analysis on ECG data associated with a patient. As one example, the electronic processor 300 executes the application 360 to determine abnormal cardiac pressures (e.g., elevated pulmonary capillary wedge pressure(s)) in patients with HF, as described in greater detail herein.

As also illustrated in FIG. 3, the memory 305 may store a learning engine 365 and a model database 370. In some embodiments, the learning engine 365 develops one or more models using one or more machine learning functions. Machine learning functions are generally functions that allow a computer application to learn without being explicitly programmed. In particular, the learning engine 365 is configured to develop an algorithm or model based on training data. For example, to perform supervised learning, the training data includes example inputs and corresponding desired (for example, actual) outputs, and the learning engine progressively develops a model (for example, a classification or detection model, etc.) that maps inputs to the outputs included in the training data. Machine learning performed by the learning engine 365 may be performed using various types of methods and mechanisms including, but not limited to, decision tree learning, association rule learning, artificial neural networks, inductive logic programming, support vector machines, clustering, Bayesian networks, reinforcement learning, representation learning, similarity and metric learning, sparse dictionary learning, genetic algorithms, etc. These approaches allow the learning engine 365 to ingest, parse, and understand data and progressively refine models for data analytics, including medical data and imaging analytics.

Models generated by the learning engine 365 can be stored in the model database 370. In some embodiments, the models generated by the learning engine 365 can perform one or more cardiac pressure analysis or function. As one example, a model stored in the model database 370 may be used to infer a probability that a patient's cardiac pressure satisfies (or otherwise meets) an exacerbation condition, as described in greater detail herein. A model stored in the model database 370 can be a convolutional neural network (“CNN”), such as, e.g., a DenseNet CNN.

As noted above, the model(s) stored in the model database 370 may be developed using training data 375 (also stored in the memory 305). In some configurations, the training data 375 includes electrocardiograme example, the training data 375 may include electrocardiogramd a corresponding classification label indicating whether the electrocardiogramdicative of an onset of a heart failure exacerbation. As illustrated in FIG. 3, the model database 370 and the training data 375 is included in the memory 305 of the server 205. It should be understood, however, that, in some embodiments, the model database 370, the training data 375, or a combination thereof is included in a separate device accessible by the server 205 (included in the server 205 or external to the server 205).

Returning to FIG. 2, the ECG device 115 collects ECG data associated with a patient. For example, the ECG device 115 can measure and record electrical signals of the heart for an associated patient and present the electrical signals in a wave-like pattern (as ECG data). Although not illustrated in FIG. 2, the ECG device 115 may include similar components as the server 205, such as electronic processor (e.g., a microprocessor, an ASIC, or another suitable electronic device), a memory (e.g., a non-transitory, computer-readable storage medium), a communication interface, such as a transceiver, for communicating over the communication network 230 and, optionally, one or more additional communication networks or connections. Alternatively, or in addition, the ECG device 115 may include additional, different, or fewer components than those illustrated and described with respect to the server 205. As one example, the ECG device 115 may include one or more electrodes, connecting wires, amplifiers, etc. for measuring electrical signals of a heart for an associated patient.

The ECG device 115 can transmit the ECG data over the communication network 230 to a remote device, such as, e.g., the server 205, the user device 210, another remote device, or a combination thereof. Alternatively, or in addition, the ECG device 115 can store the ECG data locally (e.g., in a memory of the ECG device 115). The ECG device 115 may continuously collect and transmit the ECG data in real time (or near real time) as the ECG device 115 actively measures and records the electrical signals of a patient. Alternatively, or in addition, the ECG device 115 may periodically (or intermittently) collect and transmit the ECG data based on, e.g., a transmission schedule (e.g., every five minutes, every hour, every thirty seconds, etc.). Alternatively, or in addition, the ECG device 115 can transmit the ECG data at the end of a data collection session (e.g., after completing an ECG test) for a patient. The ECG device 115 can be a portable ECG device 115, such as, e.g., a smart wearable, a patch monitor, or the like.

The user device 210 can include a computing device, such as a desktop computer, a laptop computer, a tablet computer, a terminal, a smart telephone, a smart television, a smart wearable, or another suitable computing device that interfaces with a user. Although not illustrated in FIG. 2, the user device 210 may include similar components as the server 205, such as electronic processor (e.g., a microprocessor, an ASIC, or another suitable electronic device), a memory (e.g., a non-transitory, computer-readable storage medium), a communication interface, such as a transceiver, for communicating over the communication network 230 and, optionally, one or more additional communication networks or connections. As one example, to communicate with the server 205 (or another component of the system 200), the user device 210 may store a browser application or a dedicated software application executable by an electronic processor. The system 200 is described herein as providing, among other things, a cardiac pressure monitoring service for patients with HF through the server 205. However, in other embodiments, the functionality described herein as being performed by the server 205 may be locally performed by the user device 210. For example, in some embodiments, the user device 210 may store the application 360, the learning engine 365, the model database 370, the training data 375, or a combination thereof.

In the illustrated example of FIG. 2, the user device 210 may also include a human-machine interface 385 for interacting with a user. The human-machine interface 385 may include one or more input devices, one or more output devices, or a combination thereof. Accordingly, in some embodiments, the human-machine interface 385 allows a user to interact with (e.g., provide input to and receive output from) the user device 210. For example, the human-machine interface 385 may include a keyboard, a cursor-control device (e.g., a mouse), a touch screen, a scroll ball, a mechanical button, a display device (e.g., a liquid crystal display (“LCD”)), a printer, a speaker, a microphone, or a combination thereof. As illustrated in FIG. 2, in some embodiments, the human-machine interface 385 includes a display device 390. The display device 390 may be included in the same housing as the user device 210 or may communicate with the user device 210 over one or more wired or wireless connections. As one example, in some embodiments, the display device 290 is a touchscreen included in a laptop computer or a tablet computer. In other embodiments, the display device 290 is a monitor, a television, or a projector coupled to a terminal, desktop computer, or the like via one or more cables.

A user may use the user device 210 to interact with the application 260. As one example, a user may interact with the application 260 to initiate an ECG test to collect ECG data and perform a cardiac pressure analysis on the collected ECG data, as described in greater detail herein. As also described in greater detail herein, a user may use the user device 210 to interact with a cardiac report associated with a performance of a cardiac pressure analysis. As one example, a user may view the cardiac report via the display device 290 of the user device 210. Alternatively, or in addition, a user can receive an alert or notification via the user device 210 indicating an abnormal cardiac pressure, as described in greater detail herein.

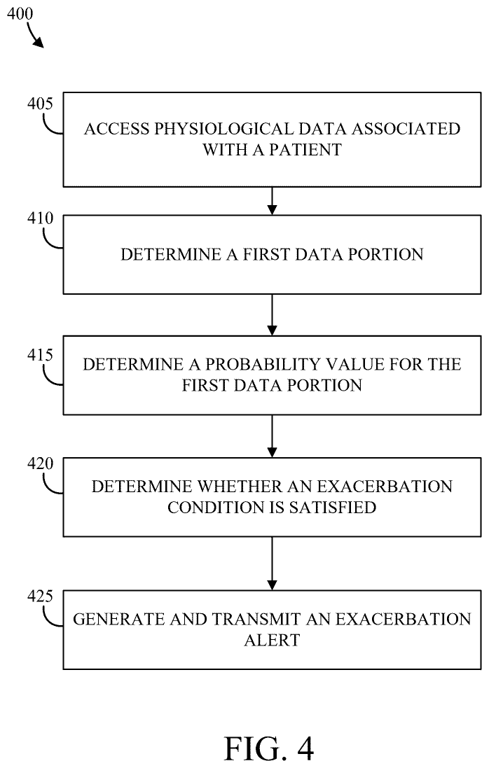

FIG. 4 is a flowchart illustrating a method 400 for detecting abnormal cardiac pressures in patients with HF performed by the system 200 according to some embodiments. The method 400 is described as being performed by the server 210 and, in particular the application 260 as executed by the electronic processor 300. However, as noted above, the functionality described with respect to the method 400 may be performed by other devices, such as the user device 205, or distributed among a plurality of devices, such as a plurality of servers included in a cloud service.

As illustrated in FIG. 4, the method 400 includes accessing physiological data associated with a patient (at block 405). In some configurations, the electronic processor 300 can receive (or access) the physiological data continuously. Alternatively, or in addition, in some configurations, the electronic processor 300 can receive (or access) the physiological data intermittently. In some configurations, the physiological data includes the ECG data collected via the ECG device 215. In such configurations, the electronic processor 300 receives (or accesses) the ECG data (as the physiological data) from the ECG device 215 over the communication network 230. In some configurations, the physiological data may include single-lead ECG data (e.g., ECG telemetry data).

As one example, where the ECG device 210 is a portable ECG monitoring device, for which a patch is placed on a patient's chest to record a signal equivalent to Lead I of the 12-lead electrocardiogram. The ECG device 210 can acquire the signal at a sampling rate of, e.g., 256 Hz with a resolution of 12 bit. Alternatively, or in addition, in some configurations, the electronic processor 300 receives (or accesses) the physiological data from another component or device included in the system 200, such as, e.g., the user device 210. Alternatively, or in addition, in some configurations, the physiological data may be locally stored in, e.g., the memory 305. In such configurations, the electronic processor 300 accesses the physiological data from the memory 305.

As illustrated in FIG. 4, the electronic processor 300 can determine a first data portion of the physiological data (at block 410). In some configurations, the electronic processor 300 pre-processes (or “cleans”) the physiological data. As one example, the electronic processor 300 can exclude the initial 60 minutes of the physiological data to avoid possible noise during sensor mounting on a patient's body. Additionally, the electronic processor 300 can exclude the same duration at the end of the recording (e.g., the physiological data), before the sensor removal. After removing the initial segment and the end segment, the electronic processor 300 may then preprocess the ECG signal (e.g., the physiological data). As one example, the physiological data can be a continuous stream of approximately 12 hours of an ECG signal for each patient. The electronic processor 300 can preprocess the physiological data in non-overlapping segments (e.g., five-minute segments). For each 5-minute segment, the electronic processor 300 can “clean” the physiological data from slow drift and/or DC offset using, e.g., a high-pass 5^{th} order Butterworth filter with a cut-off frequency at approximately 0.5 Hz. Alternatively, or in addition, the electronic processor 300 can eliminate the powerline noise with a notch filter at approximately 50 Hz.

In some configurations, the electronic processor 300 pre-processes (or “cleans”) the physiological data received at block 405. The electronic processor 300 may pre-process the physiological data by determining a set of data segments included in the physiological data. As noted above, in some configurations, the set of data segments includes a series of non-overlapping time windows included in the physiological data (e.g., non-overlapping 5-minute segments). The electronic processor 300 can also determine a set of data portions from the set of data segments, where each data portion is included in a corresponding data segment and is a representative signal of the corresponding data segment. As noted above, in some configurations, a data portion includes a predetermined or fixed window of data from the corresponding data segment (e.g., a ten-second window of data). Accordingly, the data portion may be associated with (from) a corresponding data segment included in the physiological data. For example, the first data portion may correspond with or originate from a first data segment.

In some configurations, the electronic processor 300 may determine a data portion (e.g., the first data portion) based on a signal-quality-index (“SQI”) condition. The SQI condition can refer to how noisy the physiological data is (e.g., an amount of noise). The amount of noise in the cleaned signal is estimated by calculating the SQI of the cleaned signal using, e.g., an ECG signal processing library. The electronic processor 300 can calculate the SQI (e.g., range: [0-1], noisy signals have lower SQI) for each beat of the ECG signal (e.g., the physiological data) by comparing the variation of that beat from the average beat in that 5-minute segment. As an example, the electronic processor 300 can use a threshold of 0.5 as the SQI condition to extract (or determine) a 10-second window (as the data portion) with SQI higher than 0.5, where the 10-second window is a representative signal for the 5-minute interval.

Accordingly, in some configurations, the electronic processor 300 determines (or extracts) a plurality of data portions for the physiological data (e.g., a data portion for each data segment). After determining the one or more data portions associated with the physiological data, the electronic processor 300 can determine a probability value for each data portion (at block 415). A probability value for a data portion may represent or indicate a probability that the data portion is associated with an exacerbation event (e.g., an onset of a heart failure exacerbation for the patient).

In some configurations, the electronic processor 300 determines a probability value for each data portion using a model developed via machine learning using training data (e.g., the training data 375). In such configurations, the electronic processor 300 accesses one or more models stored in the model database 370. The electronic processor 300 can then apply the one or more models to a data portion to determine a probability value associated with that data portion. As noted herein, the model(s) stored in the model database 370 may be developed using machine learning using training data, such as, e.g., electrocardiogramrelated to the current patient). Accordingly, in some configurations, the electronic processor 300 implements one or more models to infer or detect abnormal cardiac pressures in patients with HF.

After determining a set of probability values for the physiological data (at block 415), the electronic processor 300 can determine whether an exacerbation condition is satisfied based on the set of probability values (at block 420). The exacerbation condition can be associated with an abnormal cardiac pressure (e.g., an elevated pulmonary capillary wedge pressure). In some configurations, the exacerbation condition can be a predetermined threshold, such as, e.g., approximately 18 mmHg. Accordingly, as one example, an 18 mmHg threshold (as the exacerbation condition) can be used to indicate the onset of a heart failure exacerbation in patients with a pre-existing congestive heart failure diagnosis.

The electronic processor 300 may compare the set of probability values to the exacerbation condition to determine whether the exacerbation condition is satisfied (e.g., the set of probability values indicates an onset of a heart failure exacerbation). As one example, the electronic processor 300 can compare a probability value to the exacerbation condition (e.g., the 18 mmHg threshold). Following this example, the electronic processor 300 can determine that the probability value satisfies the exacerbation condition (e.g., indicating an abnormal or elevated cardiac pressure) when the probability value is greater than or equal to the exacerbation condition (e.g., occurrence of an exacerbation event). The electronic processor 300 can determine that the probability value does not satisfy the exacerbation condition (e.g., indicating a normal or non-elevated cardiac pressure) when the probability value is less than the exacerbation condition (e.g., no occurrence of an exacerbation event).

Alternatively, or in addition, in some configurations, the electronic processor 300 may combine each probability value included in the set of probability values and compare the combined probability value to the exacerbation condition. As one example, the electronic processor 300 may determine a mean probability value based on each probability value included in the set of probability values. The electronic processor 300 can compare the mean probability value to the exacerbation condition. By combining (or using a statistical mean, median, or average of) the probability values, noise can be removed from the physiological data.

In response to determining that the exacerbation condition is satisfied (e.g., occurrence of an exacerbation event), the electronic processor 300 can generate and transmit an exacerbation alert associated with the patient (at block 425). The exacerbation alert can be associated with an abnormal or elevated cardiac pressure. The exacerbation alert can include, e.g., an indication of an exacerbation event, a cardiac pressure reading associated with the exacerbation event, a date/time identifier of the exacerbation event, physiological data associated with the exacerbation event (e.g., the data segment corresponding to the exacerbation event), etc. The electronic processor 300 can transmit the exacerbation alert to a remote device, such as, e.g., the user device 210, the ECG device 215, or another component. In response to receiving the exacerbation alert, the remote device may provide the exacerbation alert to a user, such as, e.g., the patient, a medical professional, etc. The remote device may provide the exacerbation alert as an audible alert (e.g., an alarm or buzzer, an audible reading of the exacerbation alert, etc.), a tactile alert (e.g., a vibration or vibration pattern), or a visual alert (e.g., visual display of the exacerbation alert).

As one example, the electronic processor 300 can transmit the exacerbation alert to the ECG device 215. In response to receiving the exacerbation alert, the ECG device 215 may provide the exacerbation alert to the patient being monitored by the ECG device 215 by, e.g., generating an audible alert, visually displaying the exacerbation alert via a display device of the ECG device 215, etc. As another example, the electronic processor 300 can transmit the exacerbation alert to the user device 210. In response to receiving the exacerbation alert, the user device 210 can allow a user to interact with the exacerbation alert (e.g., view data associated with the exacerbation alert or event).

Alternatively, or in addition, in some configurations, the electronic processor 300 generates a report or record associated with the physiological data of the patient. The report or record may include, e.g., a set of entries associated with each probability value for each corresponding data segment, indications of each occurrence of an exacerbation event, and the like. The report or record may be stored and accessible by a user such that a user may view and interact with the report or record.

### EXAMPLE

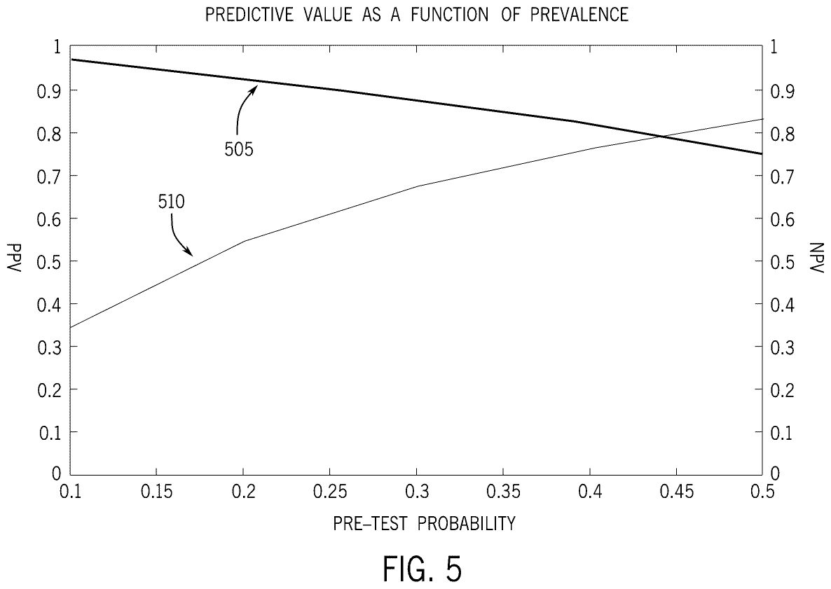

The systems and methods described herein were utilized on a cohort of 137 patients in a prospective manner. A dataset was generated by recording each patient's electrocardiogram continuously for approximately 12 hours. This occurred on the day before each patient was scheduled to undergo pulmonary artery catheterization, to measure their mPCWP. This served as the ground truth measurement for model evaluation. Using a subset of 28 patients' data, the potential of using the average probability over each patient's entire recording has been explored. When the average probability over each patient's entire recording was used, it was found that an area under the receiver operating characteristic curve (“AUROC”) of 0.83. This demonstrated that the systems and methods disclosed herein discriminate between patients with normal mPCWP and elevated mPCWP. FIG. 5 is a graph illustrating the predictive value of the systems and methods disclosed herein. In the example illustrated in FIG. 5, a single-lead ECG model for predicting elevated mPCWPs as a function of pre-test probability was implemented. Results obtained using single-lead ECG data from 27 patients referred for right heart catheterization. As illustrated, the model attains a near perfect negative predictive value (“NPV”) (represented in FIG. 5 by reference numeral 505) when the pre-test probability is low. When the pre-test probability is high, the positive predictive value (“PPV”) (represented in FIG. 5 by reference numeral 510) is greater than 0.8 (or equivalently, 80%).

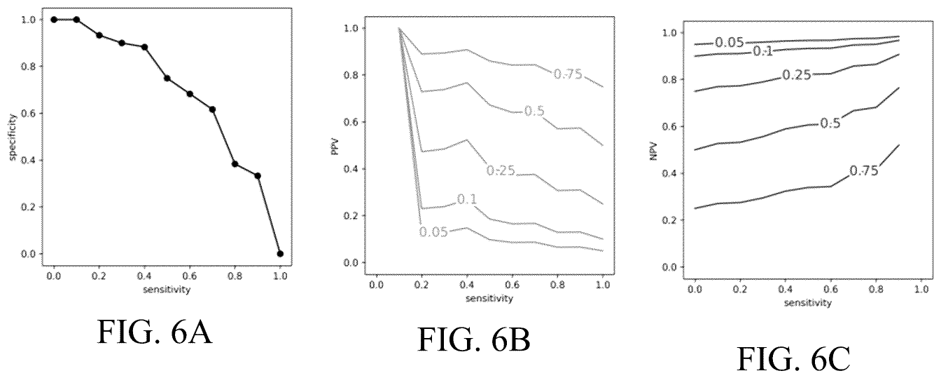

To extend the search for optimal performance, a subset of 99 patients who had a catheterization within 24 hours of ECG data collection was investigated. For these patients, the model predictions on single 10-second ECG taken from the last 5 minutes of data collection have shown to achieve 0.70 AUROC discriminative ability. The 10-second ECG is selected from the last 5 minutes of ECG data using the SQI method proposed disclosed herein. The last 5 minutes of data were assumed to be relatively closer to the time of catheterization, hence, representative of the cardiac function during the procedure. FIGS. 6A-6C illustrate the specificity, the PPV, and the NPV trends, respectively, for different sensitivity values achieved with this method, at different pre-test probabilities. As shown in FIGS. 6A-6C, for a sensitivity of 80%, the specificity achieved is about 40%. For a pre-test probability of 36% (the prevalence of elevated wedge pressure in the original development dataset), the model achieves a PPV of 43% and NPV of 79% using a decision threshold that achieves a sensitivity of 80%.

Thus, the present disclosure provides systems and methods for inferring elevated pulmonary capillary wedge pressures from single-lead electrocardiogram telemetry data.

Unless otherwise specified or limited, the terms “about” and “approximately,” as used herein with respect to a reference value, refer to variations from the reference value of ±15% or less, inclusive of the endpoints of the range. Similarly, the term “substantially,” as used herein with respect to a reference value, refers to variations from the reference value of ±5% or less, inclusive of the endpoints of the range.

Also as used herein, unless otherwise limited or defined, “or” indicates a non-exclusive list of components or operations that can be present in any variety of combinations, rather than an exclusive list of components that can be present only as alternatives to each other. For example, a list of “A, B, or C” indicates options of: A; B; C; A and B; A and C; B and C; and A, B, and C. Correspondingly, the term “or” as used herein is intended to indicate exclusive alternatives only when preceded by terms of exclusivity, such as “either,” “one of,” “only one of,” or “exactly one of.” For example, a list of “one of A, B, or C” indicates options of: A, but not B and C; B, but not A and C; and C, but not A and B. A list preceded by “one or more” (and variations thereon) and including “or” to separate listed elements indicates options of one or more of any or all of the listed elements. For example, the phrases “one or more of A, B, or C” and “at least one of A, B, or C” indicate options of: one or more A; one or more B; one or more C; one or more A and one or more B; one or more B and one or more C; one or more A and one or more C; and one or more of A, one or more of B, and one or more of C. Similarly, a list preceded by “a plurality of” (and variations thereon) and including “or” to separate listed elements indicates options of multiple instances of any or all of the listed elements. For example, the phrases “a plurality of A, B, or C” and “two or more of A, B, or C” indicate options of: A and B; B and C; A and C; and A, B, and C.

In some embodiments, aspects of the disclosure, including computerized implementations of methods according to the disclosure, can be implemented as a system, method, apparatus, or article of manufacture using standard programming or engineering techniques to produce software, firmware, hardware, or any combination thereof to control a processor device (e.g., a serial or parallel general purpose or specialized processor chip, a single- or multi-core chip, a microprocessor, a field programmable gate array, any variety of combinations of a control unit, arithmetic logic unit, and processor register, and so on), a computer (e.g., a processor device operatively coupled to a memory), or another electronically operated controller to implement aspects detailed herein. Accordingly, for example, embodiments of the disclosure can be implemented as a set of instructions, tangibly embodied on a non-transitory computer-readable media, such that a processor device can implement the instructions based upon reading the instructions from the computer-readable media. Some embodiments of the disclosure can include (or utilize) a control device such as an automation device, a special purpose or general purpose computer including various computer hardware, software, firmware, and so on, consistent with the discussion below. As specific examples, a control device can include a processor, a microcontroller, a field-programmable gate array, a programmable logic controller, logic gates etc., and other typical components that are known in the art for implementation of appropriate functionality (e.g., memory, communication systems, power sources, user interfaces and other inputs, etc.). In some embodiments, a control device can include a centralized hub controller that receives, processes and (re)transmits control signals and other data to and from other distributed control devices (e.g., an engine controller, an implement controller, a drive controller, etc.), including as part of a hub-and-spoke architecture or otherwise.

The term “article of manufacture” as used herein is intended to encompass a computer program accessible from any computer-readable device, carrier (e.g., non-transitory signals), or media (e.g., non-transitory media). For example, computer-readable media can include but are not limited to magnetic storage devices (e.g., hard disk, floppy disk, magnetic strips, and so on), optical disks (e.g., compact disk (CD), digital versatile disk (DVD), and so on), smart cards, and flash memory devices (e.g., card, stick, and so on). Additionally, it should be appreciated that a carrier wave can be employed to carry computer-readable electronic data such as those used in transmitting and receiving electronic mail or in accessing a network such as the Internet or a local area network (LAN). Those skilled in the art will recognize that many modifications may be made to these configurations without departing from the scope or spirit of the claimed subject matter.

Certain operations of methods according to the disclosure, or of systems executing those methods, may be represented schematically in the FIGS. or otherwise discussed herein. Unless otherwise specified or limited, representation in the FIGS. of particular operations in particular spatial order may not necessarily require those operations to be executed in a particular sequence corresponding to the particular spatial order. Correspondingly, certain operations represented in the FIGS., or otherwise disclosed herein, can be executed in different orders than are expressly illustrated or described, as appropriate for particular embodiments of the disclosure. Further, in some embodiments, certain operations can be executed in parallel, including by dedicated parallel processing devices, or separate computing devices configured to interoperate as part of a large system.

As used herein in the context of computer implementation, unless otherwise specified or limited, the terms “component,” “system,” “module,” “block,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

Although the present disclosure has been described with reference to preferred embodiments, workers skilled in the art will recognize that changes may be made in form and detail to the disclosed embodiments without departing from the spirit and scope of the concepts discussed herein.

1. A system for detecting abnormal cardiac pressures in patients with heart failure, the system comprising:
one or more electronic processors configured to:
receive physiological data associated with a patient;
determine, from a first data segment included in the physiological data, a first data portion satisfying a signal-quality-index (“SQI”) condition;
determine a first probability value for the first data portion with a model developed via machine learning using training data, wherein the first probability value indicates a probability that the first data portion is associated with an exacerbation event;
determine whether an exacerbation condition is satisfied based on the first probability value; and
in response to determining that the exacerbation condition is satisfied, generate and transmit an exacerbation alert associated with the patient.

2. The system of claim 1, wherein the physiological data includes electrocardiogram (“ECG”) data collected by an ECG device associated with the patient, wherein the ECG data is single-lead ECG data.

3. The system of claim 1, wherein the one or more electronic processors are configured to:
receive the physiological data continuously from a remote monitoring device associated with the patient.

4. The system of claim 1, wherein the one or more electronic processors are configured to:
receive the physiological data intermittently from a remote monitoring device associated with the patient.

5. The system of claim 1, wherein the one or more electronic processors are configured to:
determine a set of data segments included in the physiological data; and
determine a set of data portions from the set of data segments, wherein each data portion is included in a corresponding data segment and is a representative signal of the corresponding data segment,
wherein the first data segment is included in the set of data segments and the first data portion is included in the set of data portions.

6. The system of claim 5, wherein the set of data segments includes a series of non-overlapping time windows.

7. The system of claim 1, wherein the SQI condition includes a predetermined threshold of 0.5.

8. The system of claim 1, wherein the one or more electronic processors are configured to:
determine, from a second data segment included in the physiological data, a second data portion satisfying the SQI condition; and
determine a second probability value for the second data portion with the model developed via machine learning using the training data.

9. The system of claim 8, wherein the one or more electronic processors are configured to:
determine whether the exacerbation condition is satisfied based on the first probability value and the second probability value.

10. The system of claim 8, wherein the third probability value is a mean pulmonary capillary wedge pressure of the first probability value and the second probability value.

11. The system of claim 8, wherein the one or more electronic processors are configured to:
determine a third probability value based on the first probability value and the second probability value, wherein the one or more electronic processors are configured to determine whether the exacerbation condition is satisfied based on the third probability value.

12. The system of claim 11, wherein the one or more electronic processors are configured to:
determine whether the exacerbation condition is satisfied by comparing the third probability value to a pressure threshold.

13. The system of claim 12, wherein the third probability value satisfies the exacerbation condition when the third probability value exceeds the pressure threshold.

14. The system of claim 12, wherein the pressure threshold is 18 mmHg.

15. The system of claim 1, where the exacerbation condition indicates an elevated cardiac pressure that is indicative of an onset of heart failure exacerbation.

16. A method for detecting abnormal cardiac pressures in patients with heart failure, the method comprising:
receiving physiological data associated with a patient;
determining, with one or more electronic processors, a first data portion satisfying a signal-quality-index (“SQI”) condition;
determining, with the one or more electronic processors, a first probability value for the first data portion with a model relating the physiological data to cardiac pressure, wherein the first probability value indicates a probability that the first data portion is associated with an exacerbation event;
determining, with the one or more electronic processors, whether an exacerbation condition is satisfied based on the first probability value; and
in response to determining that the exacerbation condition is satisfied, generating and transmitting, with the one or more electronic processors, an exacerbation alert associated with the patient.

17. The method of claim 16, further comprising:
determining a set of data segments included in the physiological data; and
determining a set of data portions from the set of data segments, wherein each data portion is included in a corresponding data segment and is a representative signal of the corresponding data segment.

18. The method of claim 16, further comprising:
determining a plurality of data portions, wherein each data portion satisfies the SQI condition, and wherein the first data portion is included in the plurality of data portions;
determining a plurality of probability values using the model relating to the physiological data to cardiac pressure, wherein the first probability value is included in the plurality of probability values and wherein each probability value indicates a probability that each data portion is associated with a corresponding exacerbation event;
determining a combined probability value based on the plurality of probability values;
determining whether the exacerbation condition is satisfied based on the combined probability value; and
in response to determining that the exacerbation condition is satisfied, generating and transmitting the exacerbation alert associated with the patient.

19. The method of claim 18, wherein determining the combined probability value includes determining a mean pulmonary capillary wedge pressure based on the plurality of probability values.

20. The method of claim 18, wherein determining the plurality of probability values includes determining a plurality of probability values, wherein each probability value is associated with a different data portion of the plurality of data portions.
